(19) United States
(12) Patent Application Publication
(10) Pub. No.: US 2018/0158548 A1
(43) Pub. Date: Jun. 7, 2018

(54) DATA PROCESSING SYSTEMS FOR SCHEDULING WORK SHIFTS, SUCH AS PHYSICIAN WORK SHIFTS

(71) Applicant: B9 Systems, LLC, Milton, GA (US)

(72) Inventors: Soha Peter Taheri, Milton, GA (US); Tina Taheri-Khadem, Milton, GA (US)

(21) Appl. No.: 15/835,004

(22) Filed: Dec. 7, 2017

Related U.S. Application Data
(60) Provisional application No. 62/431,327, filed on Dec. 7, 2016.

(51) Int. Cl. G16H 40/20; G06Q 10/06; G06Q 10/10; G06Q 50/22

(52) U.S. Cl. CPC G16H 40/20; G06Q 50/22; G06Q 10/1093; G06Q 10/06311

(57) ABSTRACT

A computer-implemented data processing method for electronically scheduling workers to unassigned shifts based on various criteria associated with the unassigned shifts. The system receives from a first business computer, a first set of scheduling data for unassigned work shifts that are available during a scheduling period, identifies one or more users that meet particular criteria associated with each one of the unassigned work shifts, provides notification to a first that a first plurality of the unassigned work shifts are available for assignment, displays the first plurality of unassigned work shifts to the first user, receives a first request to have one or more of the first plurality of unassigned work shifts assigned to the first user, and facilitates the assignment of at least one of the one or more of the first plurality of unassigned work shifts to the first user.

### CROSS-REFERENCE TO RELATED APPLICATIONS

This application claims priority to U.S. Provisional Patent Application Ser. No. 62/431,327, filed Dec. 7, 2016, entitled, “DATA PROCESSING SYSTEMS FOR SCHEDULING WORK SHIFTS, SUCH AS PHYSICIAN WORK SHIFTS,” the disclosure of which is hereby incorporated by reference in its entirety.

### BACKGROUND

Currently, when staffing work shifts at hospitals, hospital administrators typically: (1) develop a schedule that includes the various work shifts (e.g., shifts of a predetermined number of hours) that need to be filled by physicians, advanced practitioners, nurses, and/or other personnel within an upcoming period of time (e.g., 3 or 6 months); (2) schedule available hospital staff employees to work as many of the work shifts as possible; and then (3) contact qualified independent contractors, or any other suitable individuals (including full or part-time employees of the hospital, who may wish to schedule extra shifts for additional compensation on their off days) to fill any remaining, unfilled work shifts.

The last step referenced above—the process of contacting independent contractors and other suitable individuals, and scheduling them to fill the remaining work shifts—is typically labor intensive and time consuming, and often results in poor overall staffing arrangements. As an example, a hospital secretary or other administrator may be given the task of calling independent-contractor physicians, advanced practitioners, and/or nurses to offer them various work shifts, and to update the schedule as appropriate. In doing so, the administrator may spend significant amounts of time trying to reach suitable independent contractors and may not select the best qualified independent contractors to fill the available work shifts. There is currently a need for technology to improve this process.

### SUMMARY

A computer-implemented data processing method for electronically scheduling workers to unassigned shifts based on various criteria of available workers, the method comprising (a) electronically receiving, by one or more computer processors, a first set of scheduling data associated with a first hospital comprising unassigned work shifts that are available during a scheduling period, (b) identifying, by one or more computer processors, one or more users that meet particular criteria associated with each one of the unassigned work shifts in the first set of scheduling data, (c) providing notification to a first user of the one or more users, by one or more computer processors, that a first plurality of the unassigned work shifts are available for assignment, (d) at least partially in response to receiving a request from the first user to view the first plurality of unassigned work shifts, displaying, by one or more computer processors, the first plurality of unassigned work shifts to the first user, (e) receiving from the first user, by one or more computer processors, a first request to have one or more of the first plurality of unassigned work shifts assigned to the first user, and (f) in response to receiving the request, facilitating assignment of at least one of the one or more of the first plurality of unassigned work shifts to the first user. In various embodiments, the method further comprising displaying to the first user, by one or more computer processors, an indication of compensation that the first user will receive if the first user completes one or more of the first plurality of unassigned work shifts. In some embodiments, the step of identifying one or more users that that meet particular criteria associated with each one of the unassigned work shifts in the first set of scheduling data further comprises (a) determining, by one or more computer processors, the qualifications of each user, (b) comparing, by one or more computer processors, the qualifications of each user to the minimum qualifications required for each one of the unassigned work shifts in the first set of scheduling data, and (c) selecting, by one or more computer processors, one or more users to notify about the first plurality of the unassigned work shifts. In various embodiments, the step of providing notification to a first user that a first plurality of the unassigned work shifts are available for assignment further comprises determining, by one or more computer processors, whether to provide notification to the first user based, at least in part, on one or more work performance ratings for the first user. In other embodiments, the step of providing notification to a first user that a first plurality of the unassigned work shifts are available for assignment is based on one or more characteristics associated with the first user selected from a group consisting of (a) the first user's work experience, (b) the first user's licensure, (c) ratings of the first user by their peers, (d) ratings of the first user by prior employers, (e) ratings of the first user by patients of the first user, (f) the first user's employment status, and (g) the first user's expected compensation.

In still other embodiments, the method further comprising the steps of (1) electronically receiving, by one or more computer processors, a second set of scheduling data associated with a second hospital comprising unassigned work shifts, (2) identifying, by one or more computer processors, one or more users that meet particular criteria associated with each one of the unassigned work shifts in the second set of scheduling data, (3) providing notification to the first user, by one or more computer processors, that a second plurality of the unassigned work shifts from the second set of scheduling data are available for assignment, (4) at least partially in response to receiving a request from the first user to view the second plurality of unassigned work shifts, displaying, by one or more computer processors, the second plurality of unassigned work shifts to the first user, (5) receiving from the first user, by one or more computer processors, a request to have one or more of the second plurality of unassigned work shifts assigned to the first user, and (6) in response to receiving the second request, facilitating assignment of at least one or the one or more of the second plurality of unassigned work shifts to the first user. In some of these embodiments, displaying the first and second plurality of unassigned work shifts to the first user occurs substantially simultaneously. In other embodiments, the first request and the second request are the same request.

In another embodiment of a computer-implemented data processing method for scheduling workers to unassigned work shifts, the method comprises (a) electronically receiving, by one or more computer processors, a first set of scheduling data associated with a first business indicating a first plurality of unassigned work shifts and at least one criterion for each unassigned work shift, (b) displaying to a first user, by one or more computer processors, a first plurality of unassigned work shifts that are available to the first user, (c) receiving from the first user, by one or more computer processors, a first request to have one or more of the first plurality of unassigned work shifts assigned to the first user, and (d) in response to receiving the first request, facilitating the assignment of the one or more of the first plurality of unassigned work shifts to the user. In various embodiments, the method further comprising (1) electronically receiving, by one or more processors, a second set of scheduling data associated with a second business indicating a second plurality of unassigned work shifts, (2) displaying to the first user, by one or more processors, a second indication that the second plurality of unassigned work shifts are available for assignment, (3) receiving from the first user, by one or more processors, a second request to have one or more of the second plurality of unassigned work shifts assigned to the first user, and (4) in response to receiving the second request, facilitating the assignment of the one or more of the second plurality of unassigned work shifts to the first user. In some embodiments, the method further comprises executing the step of displaying the first indication and the step of displaying the second indication simultaneously, and the first request and the second request are the same request. In other embodiments, the method further comprises determining whether to display the first set of scheduling data to the first user based, at least in part, on one or more work performance ratings for the first user. In various embodiments, the method further comprises determining whether to display the second set of scheduling data to the user based, at least in part, on one or more work performance ratings for the first user. In still other embodiments, the method further comprises displaying to the first user, by one or more processors, an indication of specific compensation that the first user will receive if the user completes one or more of the unassigned work shifts. In some of these embodiments, the method further comprises determining, by one or more processors, the specific compensation based, at least in part, on one or more work performance ratings for the first user. In yet other embodiments, the method further comprises (1) determining, by one or more processors, whether the first user is qualified to work the one or more work shifts, and (2) executing the displaying step at least partially in response to determining that the first user is qualified to work the one or more work shifts.

In yet another embodiments of a computer-implemented data processing method for scheduling work shifts, the method comprises (a) receiving, by one or more computer processors, from a first business computer, a first set of scheduling data comprising first unassigned work shifts for the first business, (b) providing notification to a first user, by one or more computer processors, that a first plurality of the first unassigned work shifts are available for assignment, (c) receiving from the first user, by one or more computer processors, a request to have one or more of the first plurality of first unassigned work shifts assigned to the first user, wherein the request comprises a compensation amount that the first user is willing to accept for each of the one or more of the first plurality of first unassigned work shifts, and (d) in response to receiving the request, facilitating assignment of the one or more of the first plurality of first unassigned work shifts at least partially based on the requested compensation amount. In some embodiments, the step of facilitating assignment of the one or more of the first plurality of first unassigned work shifts is further based on a rating associated with the first user. In other embodiments, the step of providing notification to a first user that a first plurality of the unassigned work shifts are available for assignment further comprises displaying, by one or more computer processors, the first plurality of first unassigned work shifts in a calendar display. In some of these embodiments, the calendar display is integrated with the first user's electronic calendar. In other embodiments, the method further comprises (1) receiving, by one or more computer processors, from a second business computer, a second set of scheduling data comprising second unassigned work shifts for the second business, (b) providing notification to the first user, by one or more computer processors, that a second plurality of the second unassigned work shifts are available for assignment, (c) receiving from the first user, by one or more computer processors, a second request to have one or more of the second plurality of second unassigned work shifts assigned to the first user, wherein the request comprises a compensation amount for each of the one or more of the second plurality of second unassigned work shifts, and (d) in response to receiving the second request, facilitating assignment of the one or more of the second plurality of second unassigned work shifts at least partially based on the requested compensation amount.

### BRIEF DESCRIPTION OF THE DRAWINGS

Various embodiments of the invention will be described below. In the course of the description, reference will be made to the accompanying drawings, which are not necessarily drawn to scale, and wherein:

FIG. 1 depicts a Scheduling System 100 according to particular embodiments.

FIG. 2 is a schematic diagram of a computer (such as the Central Scheduling Server 110, or one or more remote computing devices 130 that is suitable for use in various embodiments of the Scheduling System shown in FIG. 1.

FIG. 3A is a flow chart of a scheduling module in accordance with various embodiments for use in the system of FIG. 1.

FIG. 3B is a flow chart of another embodiment of a scheduling module for use with the system of FIG. 1.

FIGS. 4A-4I are screen illustrations of a user interface for use by a user of the system of

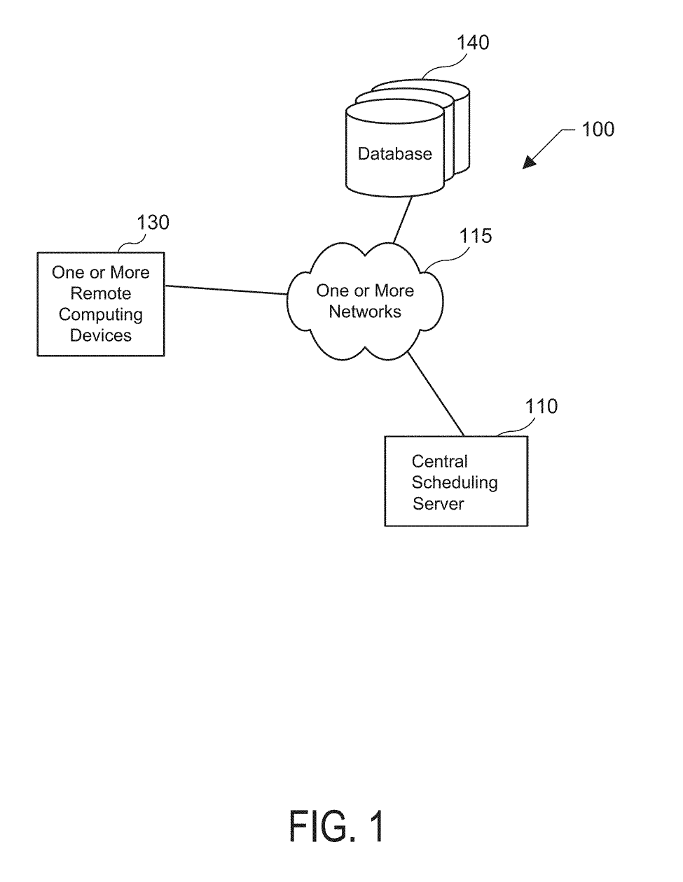

FIG. 1.

FIGS. 5A-5D are screen illustrations of a user interface for the system of FIG. 1 that illustrates the automatic assignment of work assignments to available providers;

FIGS. 6A-6F are screen illustrations of a user interface for the system of FIG. 1 that illustrates a user manually assigning work assignments to available providers.

FIGS. 7A-7J are screen illustrations of a user interface for the system of FIG. 1 that illustrates how a user swaps a work assignment with another user.

FIGS. 8A-8D are screen illustrations of a user interface for the system of FIG. 1 that illustrates how a facility creates a schedule on the system of FIG. 1.

FIG. 9A-9I are screen illustrations of a user interface for the system of FIG. 1 that illustrate how a provider requests work assignments.

### DETAILED DESCRIPTION OF VARIOUS EMBODIMENTS

Various embodiments will now be described in greater detail. It should be understood that the invention may be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

### Exemplary Technical Platforms

As will be appreciated by one skilled in the relevant field, the present systems may be, for example, embodied as a computer system, a method, or a computer program product. Accordingly, various embodiments may take the form of an entirely hardware embodiment, an entirely software embodiment, or an embodiment combining software and hardware aspects. Furthermore, particular embodiments may take the form of a computer program product stored on a computer-readable storage medium having computer-readable instructions (e.g., software) embodied in the storage medium. Various embodiments may take the form of web-implemented computer software. Any suitable computer-readable storage medium may be utilized including, for example, hard disks, compact disks, DVDs, optical storage devices, and/or magnetic storage devices.

Various embodiments are described below with reference to block diagrams and flowchart illustrations of methods, apparatuses (e.g., systems), and computer program products. It should be understood that each block of the block diagrams and flowchart illustrations, and combinations of blocks in the block diagrams and flowchart illustrations, respectively, can be implemented by a computer executing computer program instructions. These computer program instructions may be loaded onto a general purpose computer, special purpose computer, or other programmable data processing apparatus to produce a machine, such that the instructions which execute on the computer or other programmable data processing apparatus to create means for implementing the functions specified in the flowchart block or blocks.

These computer program instructions may also be stored in a computer-readable memory that can direct a computer or other programmable data processing apparatus to function in a particular manner such that the instructions stored in the computer-readable memory produce an article of manufacture that is configured for implementing the function specified in the flowchart block or blocks. The computer program instructions may also be loaded onto a computer or other programmable data processing apparatus to cause a series of operational steps to be performed on the computer or other programmable apparatus to produce a computer implemented process such that the instructions that execute on the computer or other programmable apparatus provide steps for implementing the functions specified in the flowchart block or blocks.

Accordingly, blocks of the block diagrams and flowchart illustrations support combinations of mechanisms for performing the specified functions, combinations of steps for performing the specified functions, and program instructions for performing the specified functions. It should also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and other hardware executing appropriate computer instructions.

### Overview

Data processing computer systems, according to various embodiments, are adapted to facilitate the scheduling of hospital work shifts by healthcare workers, such as independent contractor physicians, advanced practitioners, and nurses. In particular embodiments, the systems may also be used by other types of healthcare workers, such as physicians, advanced practitioners, and nurses that are on staff at a particular hospital or other types of workers.

A particular embodiment comprises one or more web servers that are collectively adapted to: (1) receive, from a first hospital computer, a first set of scheduling data indicating a first plurality of unassigned work shifts; (2) display to a user (such as an independent-contractor physician, independent-contractor advanced practitioner, or independent-contractor nurse), a first indication that the first plurality of unassigned work shifts are available for assignment; (3) receive, from the user, a request to have one or more of the first plurality of unassigned work shifts assigned to the user; and (4) in response to receiving the request, facilitate the assignment of the one or more of the first plurality of unassigned work shifts to the user.

In particular embodiments, the one or more web servers are in communication with the scheduling servers of multiple health care facilities (e.g., via the Internet or other suitable network) and the system is adapted to display (e.g., simultaneously or non-simultaneously) open work shifts for multiple health care facilities (e.g., hospitals, clinics, or other health care facilities) to the user. This may allow the user to schedule work shifts at multiple hospitals using a single software application.

In various embodiments, the system may be adapted to facilitate the rating of various users of the system by other individuals. For example, the system may be adapted to accept input, from one or more of the user's co-workers and/or patients, regarding: (1) the user's proficiency at their job; (2) the quality of the user's “bedside manner”; and/or (3) the user's ability to work well with others. The system may retrieve this information via, for example, one or more suitable surveys (e.g., online surveys).

The system may be adapted to aggregate information from multiple respective surveys from multiple respective users into a single rating score for each user and/or calculate multiple scores for an individual, each score being indicative of the individual's performance in a particular area of competence. In various embodiments, certain ratings may be kept confidential (e.g., they may not be shared with the physician), or may only be shared with a small set of individuals. In certain embodiments, some or all of the ratings may be made publicly available.

The system may, in particular embodiments, be adapted to determine and/or display an indication of compensation that a user will receive for working a particular shift at a particular health care facility. The system may calculate the offered compensation based on, for example, one or more compensation calculation algorithms, which may or may not be received from each participating health care facility. In particular embodiments, these compensation calculation algorithms may be used to calculate the offered compensation for each relevant work shift based on: (1) the desirability of the work shift (e.g., overnight work shifts may be compensated more highly than daytime work shifts); (2) one or more performance ratings associated with the user; (3) the user's seniority (e.g., total years of experience at their job, years of experience working at the particular healthcare facility, etc.); (4) the number of qualified users available to fill the position; and/or (5) any other suitable factor.

In various embodiments, the system may be adapted to time users' access to scheduling work shifts in a way that will increase the chances that the best, least-expensive users will sign up for the available shifts. In particular embodiments, the system may, for example, allow the most desirable users (e.g., the highest rated users, the most senior users, and/or the most specialized users) to access available work shifts and to schedule work within the available work shifts in one or more particular healthcare facilities earlier than other, similar users. This may be done, for example, by staggering the signup times for available work shifts. As an example, when scheduling for a particular six-month period, a hospital may allow: (1) on-staff physicians, advanced practitioners, and nurses to sign up for available shifts during a first time period (e.g., Jun. 1-5, 2017); (2) highly rated physicians, advanced practitioners, and nurses (e.g., physicians, advanced practitioners, and nurses that have been ranked, within the system, with a rating above a particular level (e.g., four out of five stars)) to sign up for any remaining available shifts during a second time period (e.g., Jun. 6-10, 2017); and (3) all remaining candidate physicians, advanced practitioners, and nurses to sign up for any remaining available shifts during a third time period (e.g., Jun. 11-30, 2017). Also, or alternatively, in various embodiments, the system may be adapted to allow healthcare facilities to specify that only individuals satisfying certain specified minimum criteria may sign up for particular work shifts (e.g., only individuals with a rating above a particular specified level may sign up for any, or certain, work shifts). This approach may help to: (1) ensure that the best-performing, least-expensive users are staffed, whenever possible; and (2) incentivize strong workplace performance by rewarding strong performance with more and/or better opportunities to work.

In various embodiments, the system may also, or alternatively, be adapted to allow users to bid on particular available work shifts. In such cases, the system may, for example, allow users to openly bid against other users up to a particular deadline in an on-line auction environment, or the system may allow users to submit their bids in a blind auction. The system may then automatically fill the work shifts based on one or specified particular criteria (e.g., the highest-rated and/or least-expensive bidding user will be assigned to each respective available work shift).

In certain embodiments, the system may be adapted to confirm that each user is properly accredited to work in a particular capacity before scheduling the user to work one or more work shifts. The system may do this, for example: (1) initially, when a user signs up for the system; (2) on a periodic basis; or (3) before scheduling any particular work shift. The system may confirm the user's accreditation by, for example, searching one or more suitable databases to confirm that the user has the proper academic accreditation to serve in the capacity needed to be executed in the work shift; that the user has not had any relevant licenses revoked by any professional governing authority; and/or that the user is current with their continuing medical education requirements. In other embodiments, the system may receive and verify this information directly from the relevant user.

### Example System Architecture

FIG. 1 is a block diagram of a Scheduling System 100 according to a particular embodiment. As may be understood from this figure, the Scheduling System 100 includes Central Scheduling Server 110, one or more networks 115, one or more remote computing devices 130 (e.g., such as a smart phone, a tablet computer, a wearable computing device, a laptop computer, a desktop computer, a remote hospital scheduling server, etc.), and one or more databases 140. In particular embodiments, the one or more networks 115 facilitate communication between the one or more remote computing devices 130, the Central Scheduling Server 110 and the one or more databases 140.

The one or more networks 115 may include any of a variety of types of wired or wireless computer networks such as the Internet, a private intranet, a mesh network, a public switch telephone network (PSTN), or any other type of network (e.g., a network that uses Bluetooth or near field communications to facilitate communication between computing devices). The communication link between the one or more remote computing devices 130 and the Central Scheduling Server 110 may be, for example, implemented via a Local Area Network (LAN) or via the Internet.

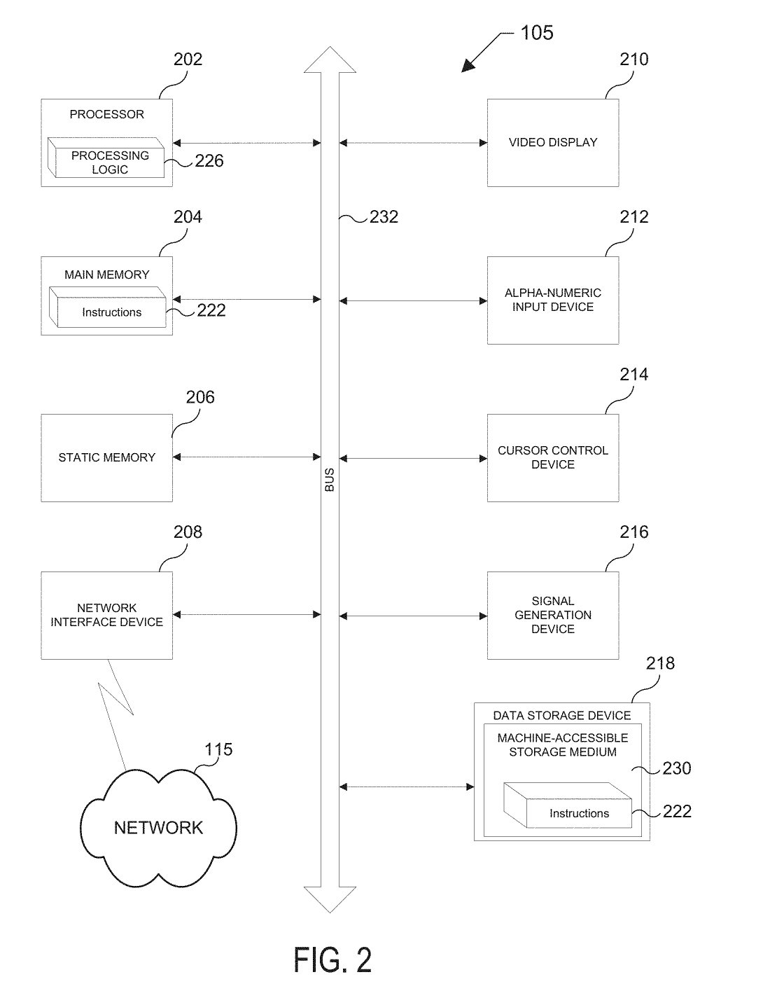

FIG. 2 illustrates a diagrammatic representation of a computer architecture 105 that can be used within the Scheduling System 100, for example, as a client computer (e.g., one of the one or more remote computing devices 130 shown in FIG. 1), or as a server computer (e.g., the Central Scheduling Server 110 shown in FIG. 1).

In particular embodiments, the computing device 105 may be connected (e.g., networked) to other computing devices via a LAN, an intranet, an extranet, and/or the Internet. As noted above, the computing device 105 may operate in the capacity of a server and/or a client computing device in a client-server network environment, and/or as a peer computing device in a peer-to-peer (or distributed) network environment. The computing device 105 may be a desktop personal computing device (PC), a tablet PC, a set-top box (STB), a Personal Digital Assistant (PDA), a cellular telephone, a web appliance, a server, a network router, a switch or bridge, or any other computing device capable of executing a set of instructions (sequential or otherwise) that specify actions to be taken by that computing device. Further, while only a single computing device is illustrated, the term “computing device” shall also be taken to include any collection of computing devices that individually or jointly execute a set (or multiple sets) of instructions to perform any one or more of the methodologies discussed herein.

Returning to FIG. 2, an exemplary computing device 105 includes a processing device 202, a main memory 204 (e.g., read-only memory (ROM), flash memory, dynamic random access memory (DRAM) such as synchronous DRAM (SDRAM) or Rambus DRAM (RDRAM), etc.), a static memory 206 (e.g., flash memory, static random access memory (SRAM), etc.), and a data storage device 218, which communicate with each other via a bus 232.

The processing device 202 represents one or more general-purpose or specific processing devices such as a microprocessor, a central processing unit, or the like. More particularly, the processing device 202 may be a complex instruction set computing (CISC) microprocessor, reduced instruction set computing (RISC) microprocessor, very long instruction word (VLIW) microprocessor, or processor implementing other instruction sets, or processors implementing a combination of instruction sets. The processing device 202 may also be one or more special-purpose processing devices such as an application specific integrated circuit (ASIC), a field programmable gate array (FPGA), a digital signal processor (DSP), network processor, or the like. The processing device 202 may be configured to execute processing logic 226 for performing various operations and steps discussed herein.

The computing device 105 may further include a network interface device 208. The computing device 105 also may include a video display unit 210 (e.g., a liquid crystal display (LCD) or a cathode ray tube (CRT)), an alphanumeric input device 212 (e.g., a keyboard), a cursor control device 214 (e.g., a mouse), and a signal generation device 216 (e.g., a speaker).

The data storage device 218 may include a non-transitory computing device-accessible storage medium 230 (also known as a non-transitory computing device-readable storage medium or a non-transitory computer-readable medium) on which is stored one or more sets of instructions (e.g., software 222) embodying any one or more of the methodologies or functions described herein. The software 222 may also reside, completely or at least partially, within the main memory 204 and/or within the processing device 202 during execution thereof by the computing device 120—the main memory 204 and the processing device 202 also constituting computing device-accessible storage media. The software 222 may further be transmitted or received over a network 115 via a network interface device 208.

While the computing device-accessible storage medium 230 is shown in an exemplary embodiment to be a single medium, the terms “computing device-accessible storage medium” and “computer-readable medium” should be understood to include a single medium or multiple media (e.g., a centralized or distributed database, and/or associated caches and servers) that store the one or more sets of instructions. The terms “computing device-accessible storage medium” and “computer-readable medium” should also be understood to include any device that is capable of storing, encoding or carrying a set of instructions for execution by the computing device and that cause the computing device to perform any one or more of the methodologies of the present invention. The terms “computing device-accessible storage medium” and “computer-readable medium” should accordingly be understood to include, but not be limited to, solid-state memories, optical and magnetic media, etc.

### Scheduling Module

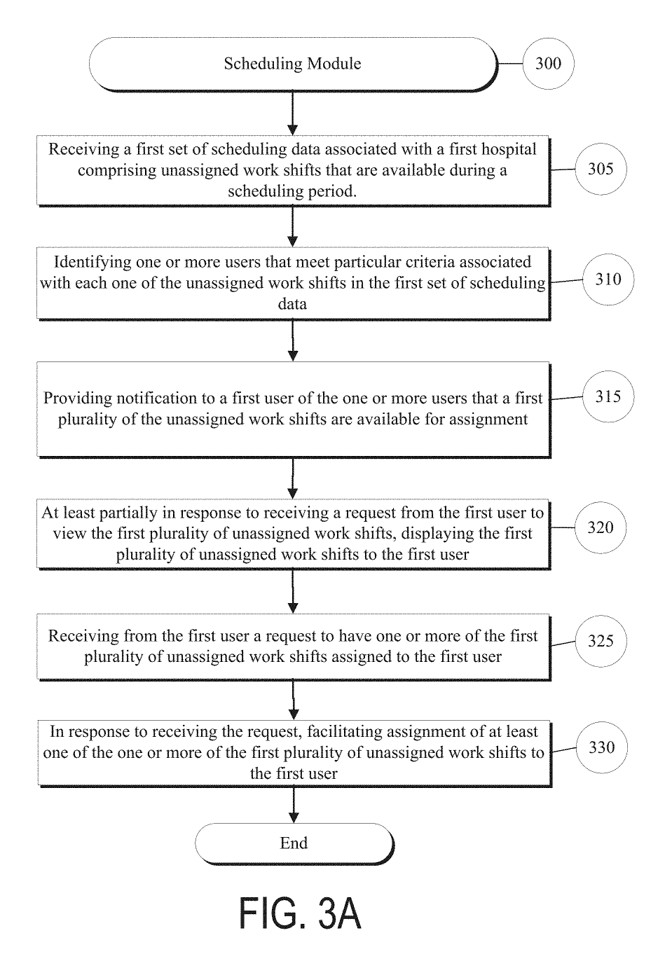

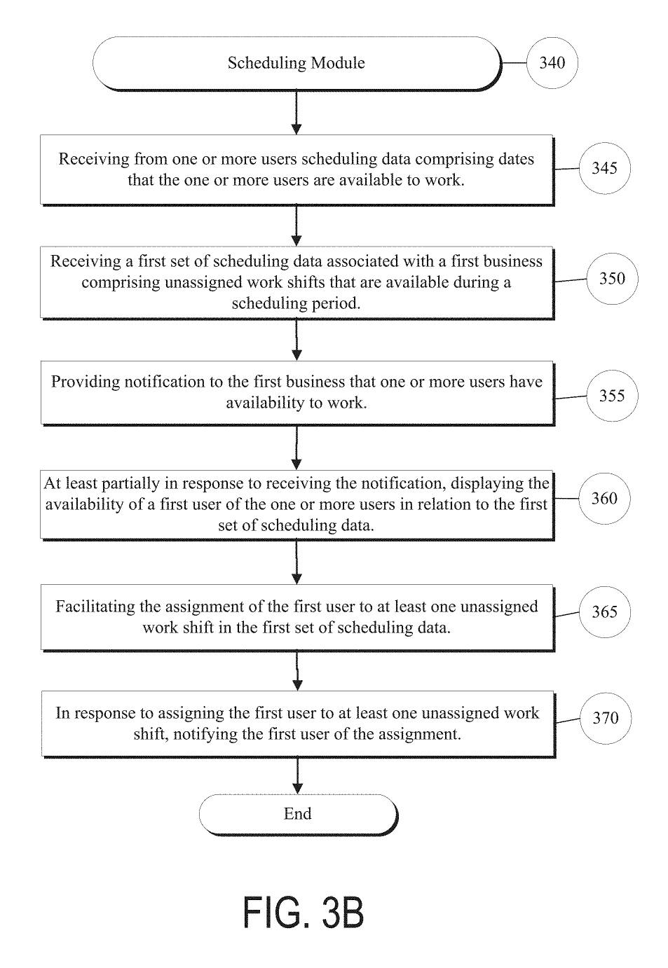

Various aspects of the system's functionality may be executed by certain system modules, including a Scheduling Module 300 (FIG. 3A) that allows users to request certain available shifts and a Scheduling Module 340 (FIG. 3B) that allows users to submit their availability to the system as further described with respect to FIGS. 6A-6F. These modules are discussed in greater detail below. Although these modules are presented as a series of steps, it should be understood in light of this disclosure that various embodiments of the modules described herein may perform the steps described below in an order other than in which it is presented. In still other embodiments, the modules may omit certain steps described below. In various other embodiments, the modules described herein may perform steps in addition to those described in the modules and/or instead of those steps described in the modules. In still other embodiments, the modules described herein may perform some steps from one module and other steps from the other module.

### First Embodiment of the Scheduling Module

Electronically Receiving a First Set of Scheduling Data Associated with a First Hospital Comprising Unassigned Work Shifts That are Available During a Scheduling Period.

In particular embodiments, when executing the Scheduling Module 300 shown in FIG. 3A, the system begins, at Step 305, by receiving a first set of scheduling data. The scheduling data may be received from any number of businesses, not for profit organizations, etc. For example, in the case of a hospital, the hospital may have full-time staff that is assigned to 75% of work shifts available at the hospital. The remaining shifts may be filled by contract employees. This enables the hospital to reduce costs by not over staffing. Following the example of a hospital, a first scheduling server, which is associated with a first hospital, and a second scheduling server, which is associated with a second hospital, are connected, via a suitable network (e.g., the Internet or any other suitable network) to a central scheduling server 110, which may, for example, be a central web server. In advance of an upcoming particular scheduling period (e.g., a particular three-month scheduling period), a scheduling representative of the first hospital and a scheduling representative of the second hospital will generate a list of work shifts that need to be filled within that scheduling period. For example, in various embodiments, each hospital may have a certain number of work shifts for various positions that must be filled each day of a scheduling period. Thus, the available work shift information, which may include, the day, time, qualifications to work the position, compensation offered, etc. is transmitted via a data file to the central scheduling server.

Identifying One or More Users That Meet Particular Criteria Associated with Each One of the Unassigned Work Shifts in the First Set of Scheduling Data

Next, at Step 310, the system identifies one or more users of the system that meet particular criteria associated with each one of the unassigned work shifts received in the scheduling data from one or more enterprises (e.g., hospital, business, not-for-profit, etc.). Continuing with the hospital example, the system may have one or more registered users. When a user registers with the system, the user is required to establish an account on the system and enter all of their pertinent information including, but not limited to, name, address, telephone number, work history, education history, certifications and associated license information (e.g., board certification numbers, registered nurse certificate numbers, etc.). In various embodiments, the system upon receiving user registration information then verifies the accuracy of the submitted information. Verification may be done automatically by linking to State Boards, national board, certifying entities, etc. In other embodiments, the information may be manually verified. In still other embodiments, the information supplied by a user may be both automatically and manually verified.

In certain embodiments, the system may be adapted to confirm that each user is properly accredited to work in a particular capacity before providing notification to the user or scheduling the user to work one or more work shifts. The system may do this, for example: (1) initially, when a user signs up for the system; (2) on a periodic basis; or (3) before scheduling any particular work shift. The system may confirm the user's accreditation by, for example, searching one or more suitable databases to confirm that the user has the proper academic accreditation to serve in the capacity needed to be executed in the work shift; that the user has not had any relevant licenses revoked by any professional governing authority; and/or that the user is current with their continuing medical education requirements. In other embodiments, the system may receive and verify this information directly from the relevant user.

In particular embodiments, once the user information has been verified, the user's account may be activated so that the user may be selected (e.g. identified by the system) for one or more listed vacancies. In various embodiments, the system may be configured to identify one or more users that meet particular criteria associated with an unassigned work shift when a user meets the minimum certification requirement for the unassigned work shift. For example, when an unassigned work shift is for a shift in an emergency room, the system may identify all boarded doctors since any boarded doctor may work a shift in an emergency room. In another example, for an unassigned work shift in radiology, the system may identify all boarded radiologists as the one or more users.

In some embodiments, the system may identify the one or more users based on one or more criteria. For example, for the emergency room shift, the first criteria may be a boarded doctor and the second criteria may be having a 4 star rating from patients. Thus, only boarded doctors that have at least a 4 star rating would be identified by the system for the unassigned work shift. It should be understood that the system may identify the one or more users based on any one of a number of available criteria. For example, the system may be configured to indemnity one or more users based on the minimum compensation that the user requires to fill an unassigned work shift. Thus, if the compensation for a work shift is below the minimum compensation that a user is willing to accept, the user will not be identified by the system as a potential candidate for the unassigned work shift.

Providing Notification to a First User of the One or More Users That a First Plurality of the Unassigned Work Shifts are Available for Assignment

In various embodiments, the Scheduling System 100, at Step 315, provides notification to a first user of the one or more users that a first plurality of the unassigned work shifts are available for assignment. The first user is selected from the identified one or more users who meet particular criteria associated with each of the unassigned work shifts. In various embodiments, the first plurality of the unassigned work shifts may comprise at least one work shift that the first user qualifies for. In other embodiments, the first plurality of the unassigned work shifts may comprise multiple unassigned work shifts that the first user qualifies for based on criteria set by the business.

In various embodiments, the notification may comprise one or more of sending an e-mail to the first user, sending a text message to the first user, providing a numeric notification on an application icon that one or more unassigned work shifts are available to the first user. It should be understood that there are many other suitable notifications that the system may use such as providing a telephone call to the first user. In still other embodiments, the notification may be provided by showing an indication of the number of available shifts on the icon for the program.

In various embodiments, the system may be adapted to time users' notification and/or access to scheduling work shifts in a way that will increase the chances that the best, least-expensive users will sign up for the available shifts. In particular embodiments, the system may, for example, notify and allow the most desirable users (e.g., the highest rated users, the most senior users, and/or the most specialized users) to access available work shifts and to schedule work within the available work shifts in one or more particular healthcare facilities earlier than other, similar users. This may be done, for example, by staggering the notification times and/or signup times for available work shifts. As an example, when scheduling for a particular six-month period, a hospital may allow: (1) on-staff physicians, advanced practitioners, and nurses to sign up for available shifts during a first time period (e.g., Jun. 1-5, 2017); (2) highly rated physicians, advanced practitioners, and nurses (e.g., physicians, advanced practitioners, and nurses that have been ranked, within the system, with a rating above a particular level (e.g., four out of five stars)) to sign up for any remaining available shifts during a second time period (e.g., Jun. 6-10, 2017); and (3) all remaining candidate physicians, advanced practitioners, and nurses to sign up for any remaining available shifts during a third time period (e.g., Jun. 11-30, 2017).

Also, or alternatively, in various embodiments, the system may be adapted to allow healthcare facilities to specify that only individuals satisfying certain specified minimum criteria may sign up for particular work shifts (e.g., only individuals with a rating above a particular specified level may sign up for any, or certain, work shifts). This approach may help to: (1) ensure that the best-performing, least-expensive users are staffed, whenever possible; and (2) incentivize strong workplace performance by rewarding strong performance with more and/or better opportunities to work.

In various embodiments, the system notification may, for example, include transmitting suitable electronic messages to the user when it is possible for the user to sign up for available shifts. The notification may also include, for example, a link to a secure website that the user may use to log into to view available work shifts that are available to sign up for. The website, which may be supported by the central scheduling server, may display available work shifts in any suitable manner (e.g., in the form of entries on a graphical calendar, as lists, as text, etc.). The work shifts may include work shifts for any suitable number of different health care facilities, which may, for example, allow independent-contractor healthcare workers to easily prepare cohesive, non-conflicting work schedules at multiple healthcare facilities using the system.

At Least Partially in Response to Receiving a Request from the First User to View the First Plurality of Unassigned Work Shifts, Displaying the First Plurality of Unassigned Work Shifts to the First User

In various embodiments, the system at Step 320, displays the first plurality of unassigned work shifts to the first user at least partially in response to receiving a request from the first user to view the unassigned work shifts. In various embodiments, where the notification is via a text or program notification, when the first user selects the text of program notification the program may be configured to open to display the available unassigned work shifts to the first user. In another embodiment, a software program on the first user's handheld device (e.g., tablet, smart phone, etc.) or computer may integrate with the first user's electronic calendar. In these embodiments, the available unassigned work shifts may tentatively show up on the first user's calendar to allow the first user to compare their availability to fill the unassigned work shift with their current schedule. In other embodiments, the program may have its own user interface such as a calendar view that shows the available unassigned work shifts. In some of these embodiments, the program calendar view may integrate with the electronic calendar on the handheld device so that the unassigned work shifts may be shown in reference to other appointments already scheduled by the first user.

In various embodiments, the system is adapted to display (e.g., simultaneously or non-simultaneously) open work shifts for multiple businesses (e.g., health care facilities (e.g., hospitals, clinics, or other health care facilities)) to the user. This allows users to schedule work shifts at multiple business using a single software application.

The system may, in particular embodiments, be adapted to determine and/or display an indication of compensation that a user will receive for working a particular shift at a particular health care facility. The system may calculate the offered compensation based on, for example, one or more compensation calculation algorithms, which may or may not be received from each participating health care facility. In particular embodiments, these compensation calculation algorithms may be used to calculate the offered compensation for each relevant work shift based on: (1) the desirability of the work shift (e.g., overnight work shifts may be compensated more highly than daytime work shifts); (2) one or more performance ratings associated with the user; (3) the user's seniority (e.g., total years of experience at their job, years of experience working at the particular healthcare facility, etc.); (4) the number of qualified users available to fill the position; and/or (5) any other suitable factor.

Receiving from the First User a Request to Have One or More of the First Plurality of Unassigned Work Shifts Assigned to the First User

In various embodiments, the system 100, at Step 325, is configured to receive a request from a user to have one or more of the unassigned work shifts assigned to the user. The request may be made via any suitable mode of communication. For example, in various embodiments, the user may select one or more of the unassigned work shifts that are displayed to the user in Step 320 and send a request to the system that the user wishes to accept those unassigned work shifts. In some embodiments, the user can do this by selected one or more unassigned work shifts from a list of unassigned work shifts that the user qualifies for and select an accept button on the user interface that notifies the system that the user is interested in working the selected shifts. In other embodiments, the available work shifts may be displayed on a calendar display that allows the user to select the days that the user is interested in working. In this way, if there is more than one work shift available on that day, the user may configure account settings to select the best suited work shift that is available on that day. For example, the user setting may allow the system to select one of multiple work shifts available on the selected date based on one or more of (1) the highest paying work shift available on that day, (2) the closest work location to a predefined location (e.g., the user's home address), (3) the highest rated facility, or any other suitable criteria.

In various embodiments, the system 100 may also, or alternatively, be adapted to allow a user to bid on particular available work shifts. In such cases, the system may, for example, allow users to openly bid against other users up to a particular deadline in an on-line auction environment, or the system may allow users to submit their bids in a blind auction. The system may then automatically fill the work shifts based on one or specified particular criteria (e.g., the highest-rated and/or least-expensive bidding user will be assigned to each respective available work shift).

In Response to Receiving the Request, Facilitating Assignment of at Least One of the One or More of the First Plurality of Unassigned Work Shifts to the First User

In various embodiments, the system 100, at Step 330, in response to receiving the request from the user, facilitate assignment of at least one of the one or more first plurality of unassigned work shifts to the first user. The system facilitates assignment to the user based on one or more factors that are set up by the submitter of the unassigned work shifts. For example, in the hospital example, the system may select the assigned user based on user rating, requested compensation, user availability based on other user requested work assignment, etc. Thus, if a user requests multiple work assignment from the available work assignments, the system may award assignments to maximize a particular user's ability to accept multiple available work assignments. Said another way, for a highly rated user, the system may optimize this particular user's requests so that they are assigned most or all of their requested assignments, whereas a lower rated users may only get one of their requested assignments.

### Second Embodiment of the Scheduling Module

In a second embodiment of a Scheduling Module 340, the module begins at Step 345, by electronically receiving scheduling data from one or more users that comprises dates that the one or more users are available to work. The scheduling data may also include additional information about the user such as licensure information, national provider identification number or any other information associated with the user that is necessary to allow the user to be assigned a work shift. Next at Step 350, the system 100 receives a first set of scheduling data associated with a first business (e.g., a hospital, etc.) comprising unassigned work shifts that are available during a scheduling period. For example, in a hospital setting, the unassigned work shifts would correspond to one or more work shifts for medical doctors, nurses, healthcare providers, etc.

At step 355, the system 100 provides notification to the first business that one or more users have availability to work. In various embodiments, the notification may be sent to a hospital administrator, a scheduling clerk, the staff supervisor or any other suitable party that is in charging of scheduling. At Step 360, at least partially in response to receiving the notification, the system 100 displays the availability of a first user of the one or more users in relation to the first set of scheduling data. In various embodiments, the first user data may be shown in a calendar view aligned with the first set of scheduling data, as shown in FIG. 6C.

At Step 365, the system 100 facilitates the assignment of the first user to at least one unassigned work shift in the first set of scheduling data. In various embodiments, the system 100 may automatically assign the first user to one or more unassigned work shifts in the first set of scheduling data based on one or more criteria (e.g., user rating, compensation requested, etc.) associated with the first user. In other embodiments, the system 100 may allow for manual assignment of the first user to one or more of the unassigned work shifts. Finally, at Step 370, the system 100 in response to the assignment notifies the first user of the one or more work assignments.

### Alternative Features for the Scheduling Module

In various embodiments, the system 100 may be adapted to facilitate the rating of various users of the system by other individuals. For example, the system may be adapted to accept input, from one or more of the user's co-workers and/or patients, regarding: (1) the user's proficiency at their job; (2) the quality of the user's “bedside manner”; and/or (3) the user's ability to work well with others. The system may retrieve this information via, for example, one or more suitable surveys (e.g., online surveys).

The system may be adapted to aggregate information from multiple respective surveys from multiple respective users into a single rating score for each user and/or calculate multiple scores for an individual, each score being indicative of the individual's performance in a particular area of competence. In various embodiments, certain ratings may be kept confidential (e.g., they may not be shared with the physician), or may only be shared with a small set of individuals. In certain embodiments, some or all of the ratings may be made publicly available.

### Example Operation of the System

The structure and operation of a particular system is described below. It should be understood that this discussion is intended for example purposes only and is not intended to be limiting.

User Registration with the System

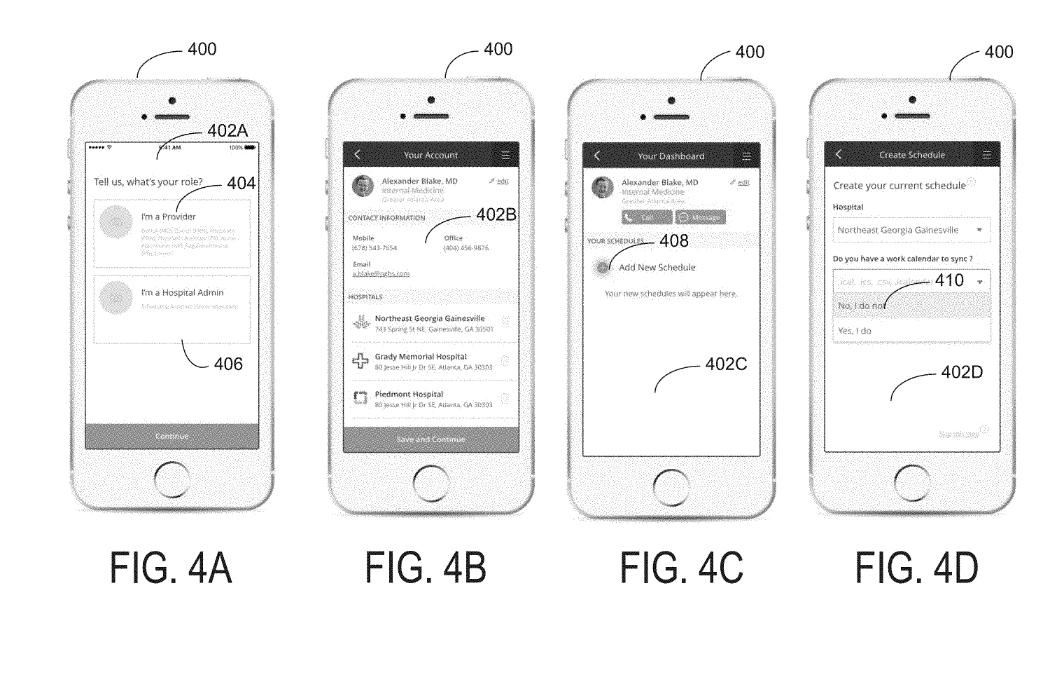

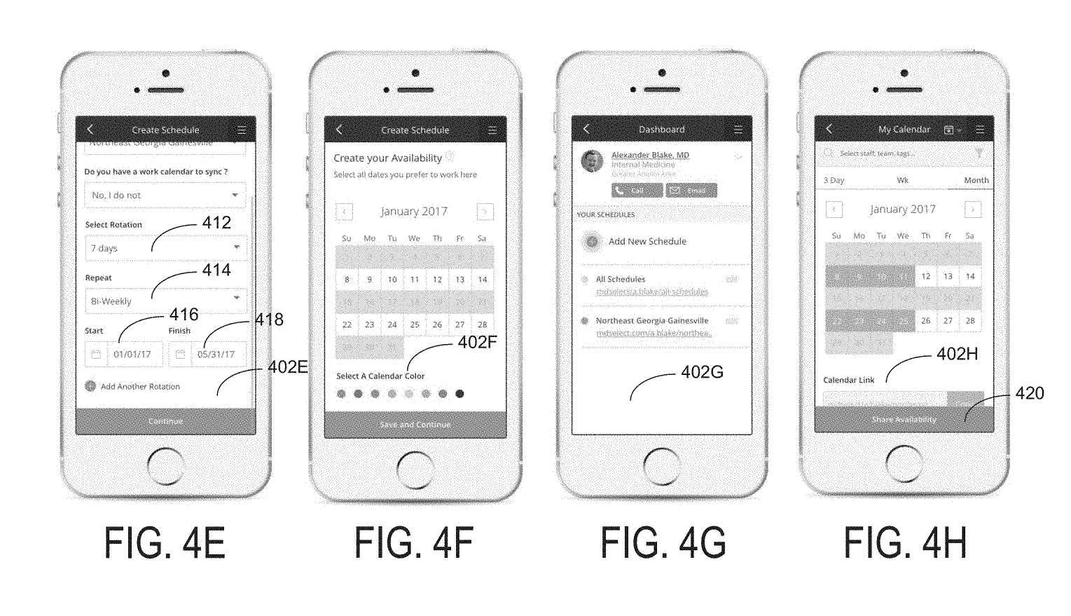

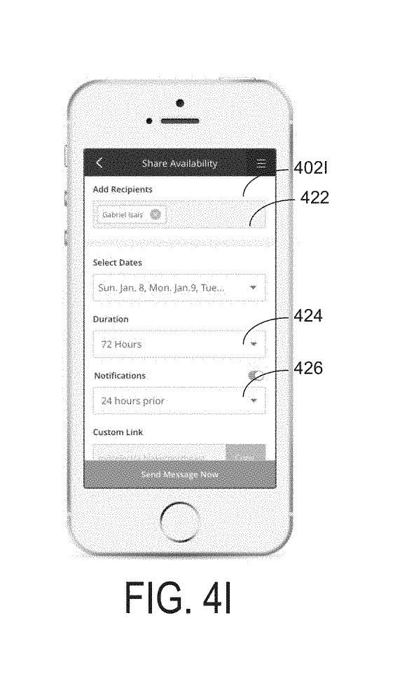

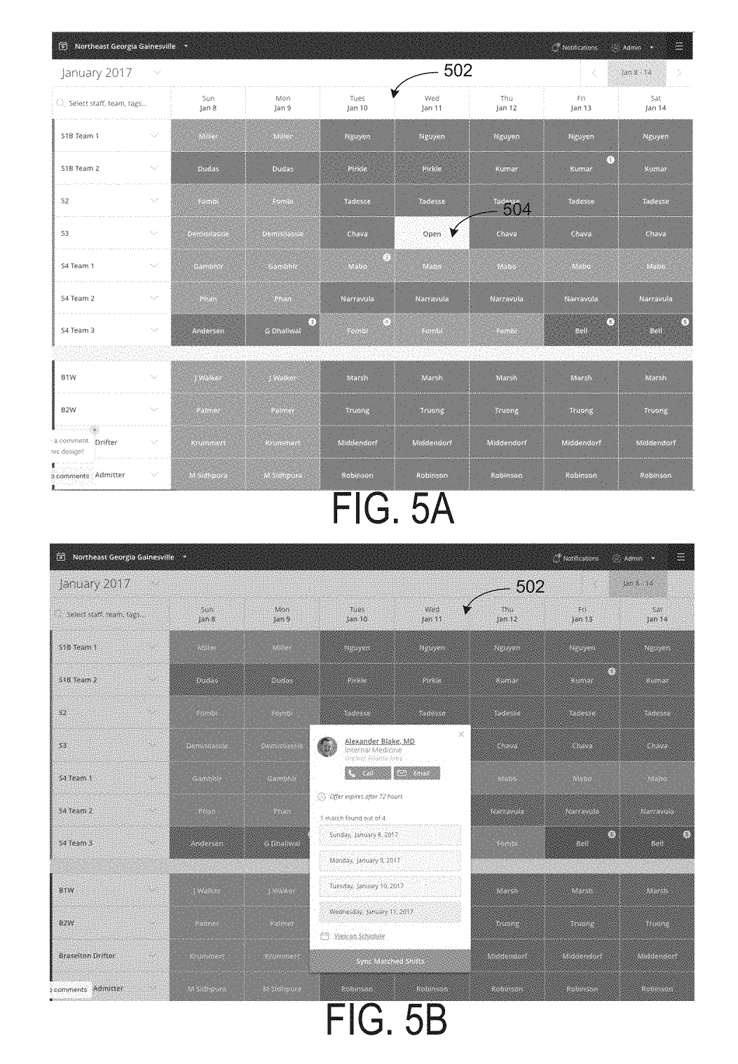

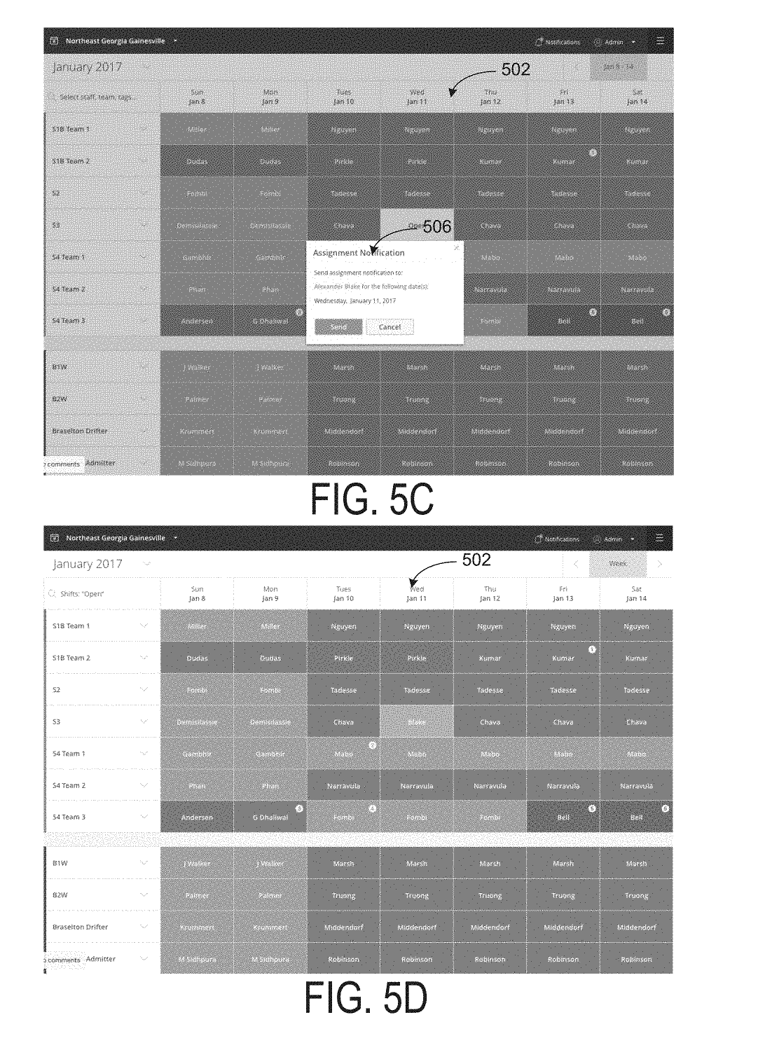

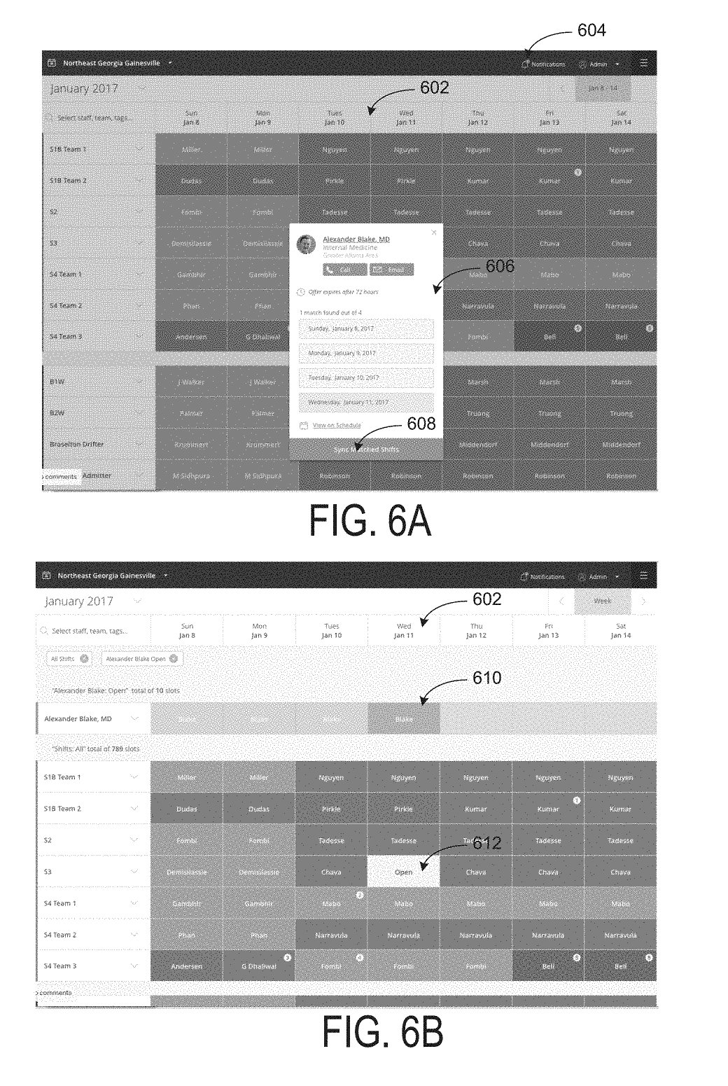

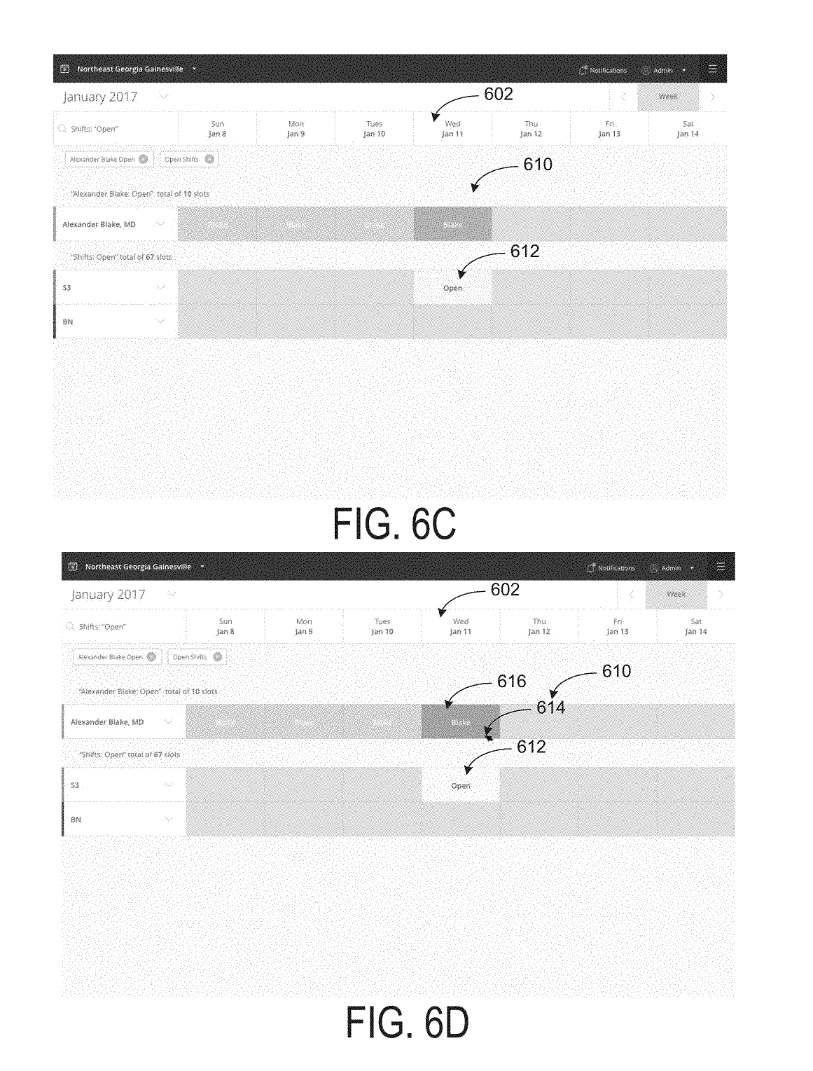

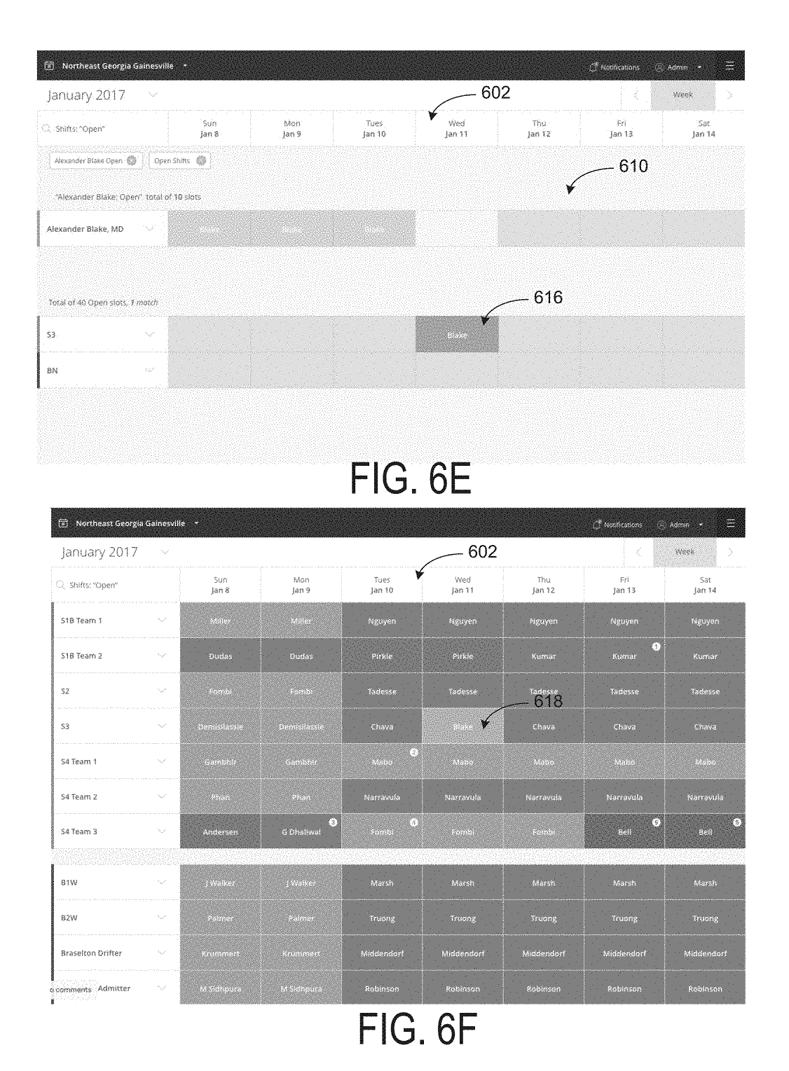

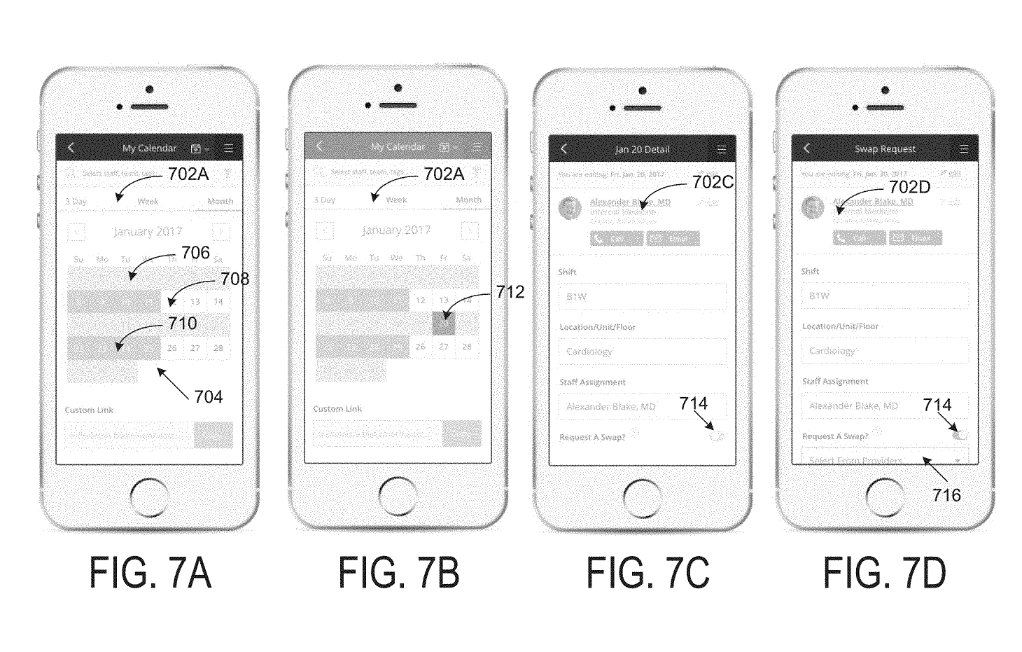

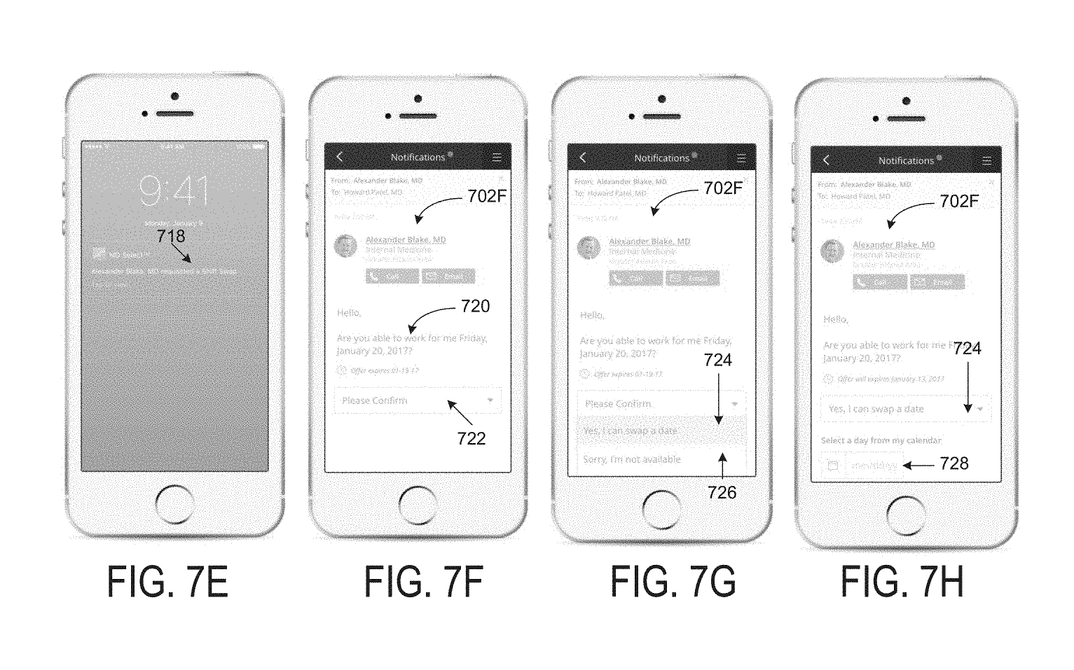

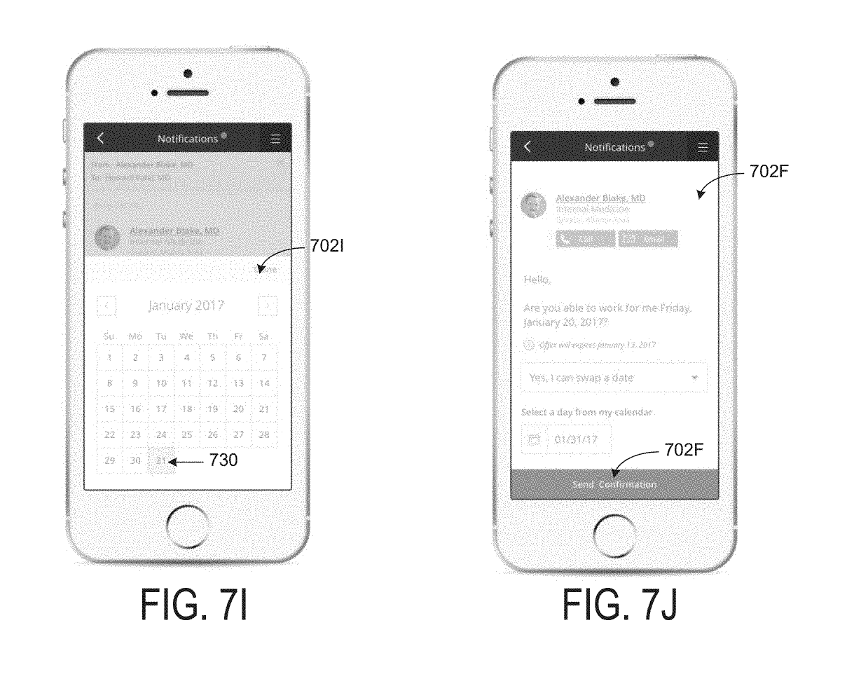

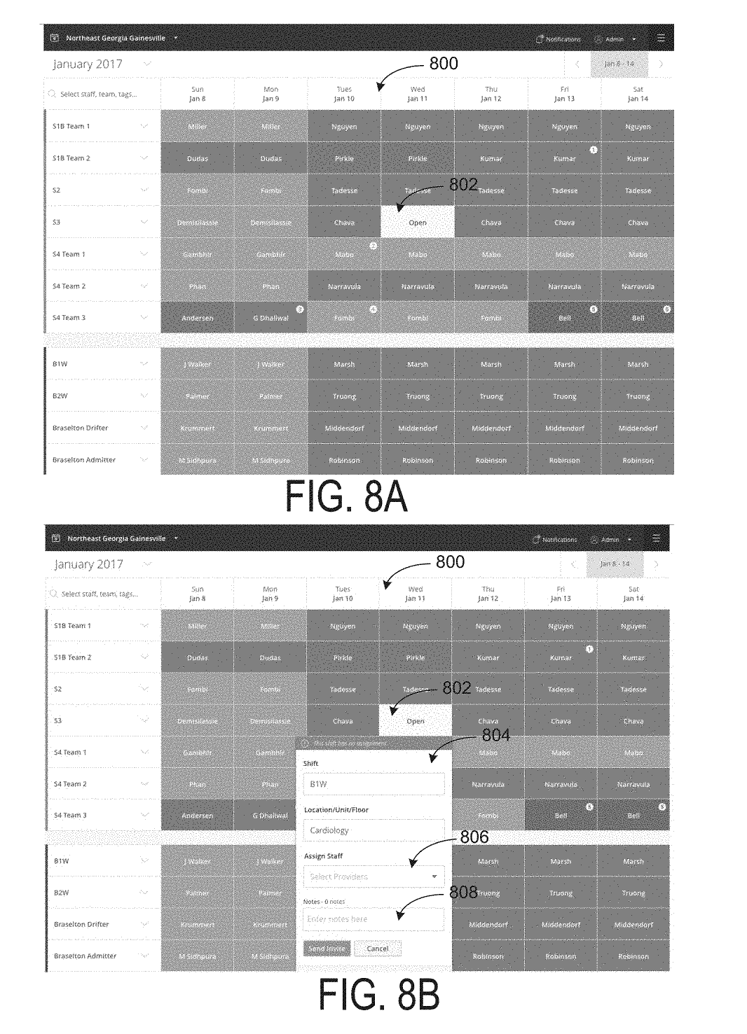

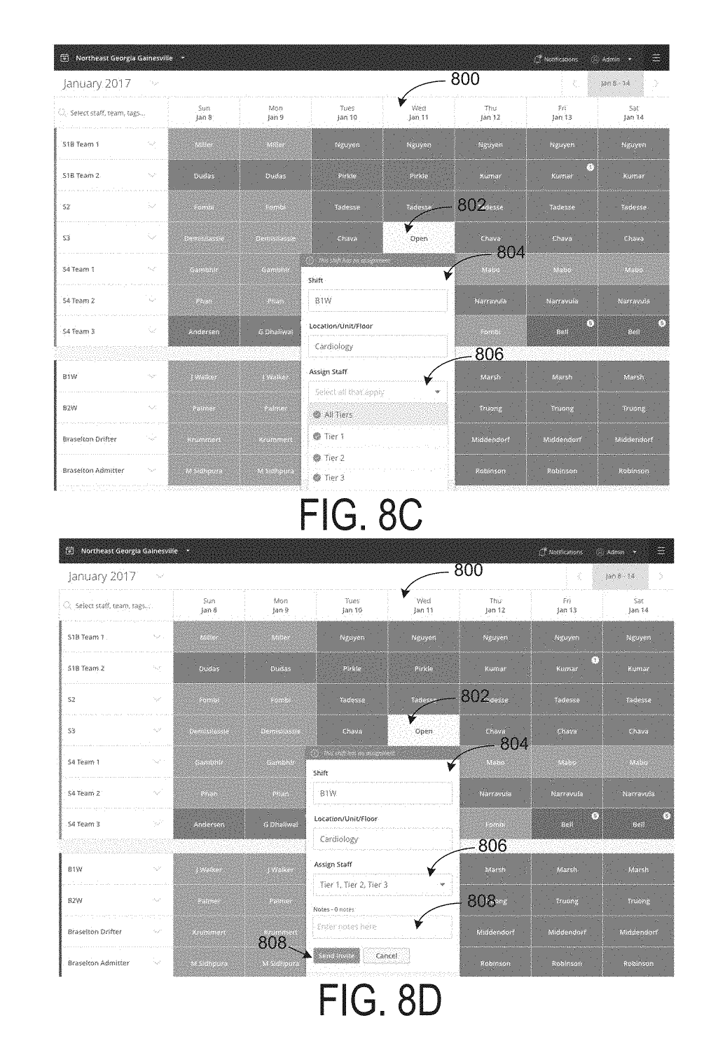

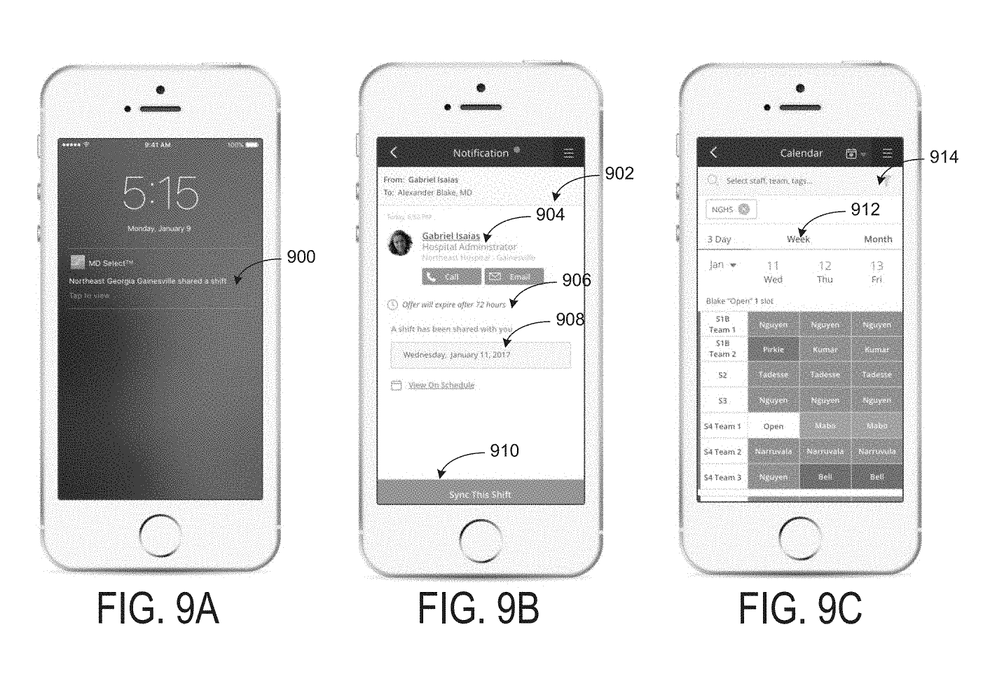

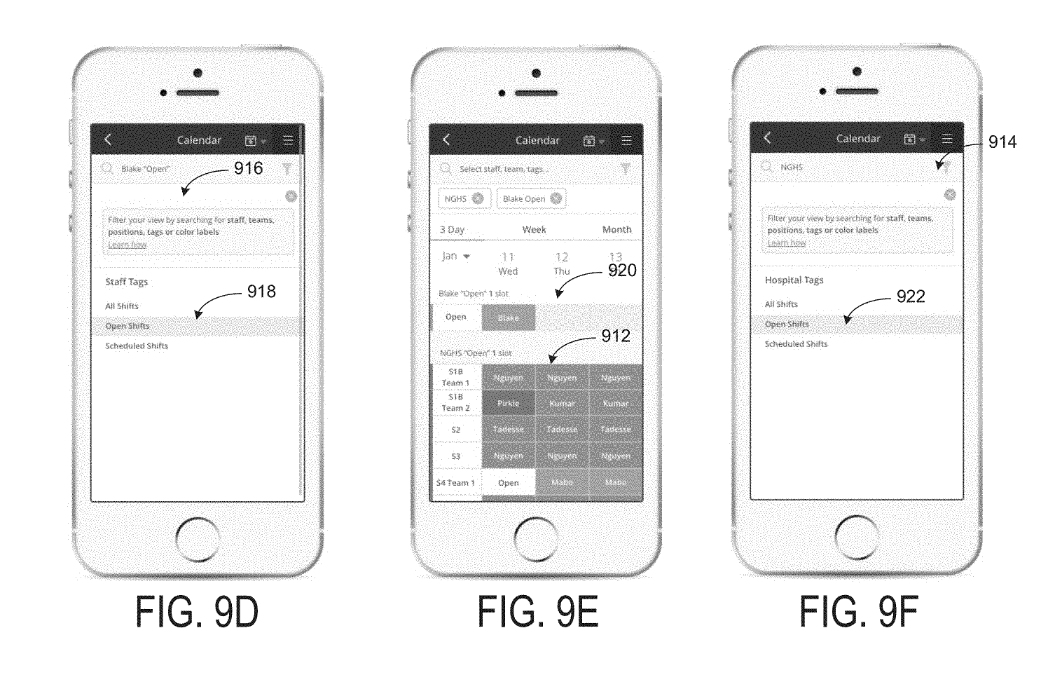

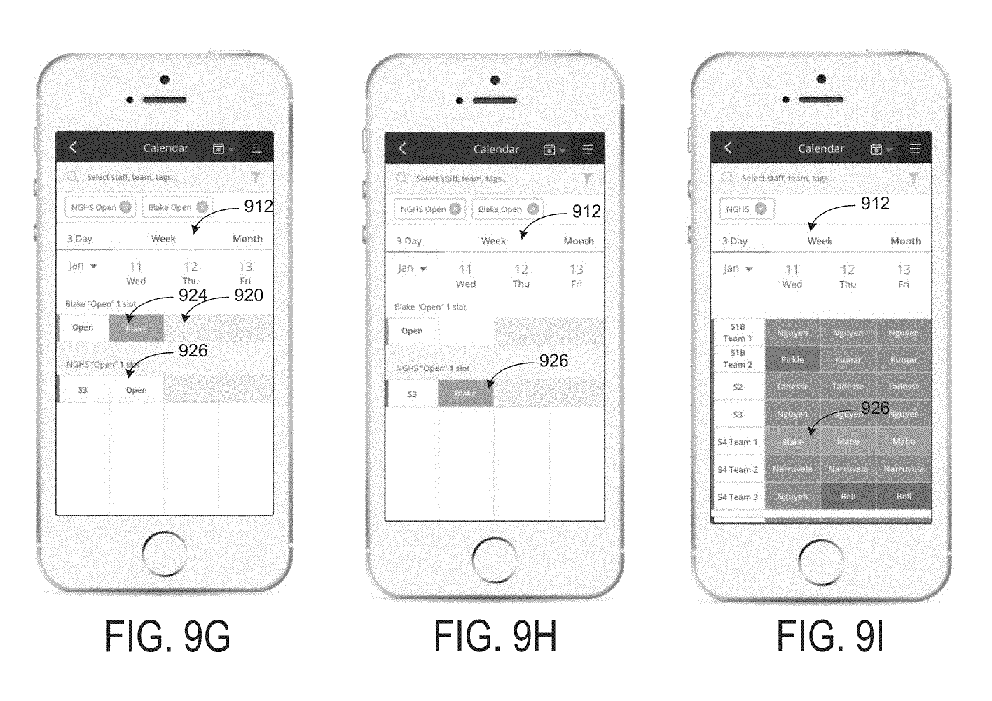

In various embodiments and referring to FIGS. 4A-4I, a user begins use of the system by downloading the application to a computing device 400. It should be understood, that in various embodiments, the user may instead register using a web application, or webpage. Once the application is downloaded to the computing device 400, selects to register with the system 100 by entering their name, address, telephone number, e-mail address. Once the basic user information is submitted, the user interface 402A provides the user with a provider item 404 or a business item 406 (e.g., a hospital, etc.) that allows the user to select their role in the system 100. In the present example, the user selects the provider item 404 indicating that they are a provider that is seeking open work shifts. The system 100 then asks the user what hospitals that they work at (e.g., what hospitals that they are credentialed at, etc.) and provides a drop down menu for the user to select from. The user may select one or more hospital that they are affiliated with. The system may also be configured to obtain licensure information, national provider identification number or any other information that is necessary from the user.

Referring to FIG. 4B, the user interface 402B then displays the user's account information. Referring to FIG. 4C, the user interface 402C next presents the user with the option to add their schedule to their account by selecting an “add new schedule” item 408. Referring to FIG. 4D, the user interface 402D next provides the user with the ability to select whether they wish to sync a work calendar with their account on system 100. In the present example, the user does not have a work calendar so they select the “no, I do not” menu item 410, which brings the user to the user interface 402 shown in FIG. 4E. Referring to FIG. 4E, the user interface 402E allows the user to select their present work rotation at menu item 412, how often the rotation repeats at menu item 414, and a start date 416 and an end date 418 for their current schedule. Next at FIG. 4F, the user interface 402F allows the user to select their current availability to work additional shifts. Once the user completes the process to add their schedule(s), the system displays a list of their current schedules at user interface 402G (Fig. 4G). The user may then share their current availability with the system 100 by selecting a “share availability” menu item 420 on user interface 402H (FIG. 4H). Once the user selects the “share availability” menu item 420, the system presents the user with user interface 402I (FIG. 4I) to allow the user to share their availability with multiple businesses (hospitals, etc.) by selecting one or more recipients at menu item 422, an indication of the duration of their availability offer at menu item 424, and a predetermined time frame for advanced notification of a work shift start at menu item 426. Some of the information, such as the duration and the prior notification may be optional information in various embodiments. At this point, the system 100 receives the user availability information.

Automatic and Manual Work Shift Assignments

In particular embodiments, a first scheduling server, which is associated with a first hospital, and a second scheduling server, which is associated with a second hospital, are connected, via a suitable network (e.g., the Internet or any other suitable network) to a central scheduling server, which may, for example, be a central web server. In advance of an upcoming particular scheduling period (e.g., a particular three-month scheduling period), a scheduling representative of a first hospital generates a list of work shifts that need to be filled within that scheduling period. Each work shift identifies the start and stop time of the work shift, the types of healthcare professionals needed to fulfill each work shift (e.g., general surgeon, anesthesiologist, surgical assistant, etc.) and the location where the work is to be performed (e.g., Operating Room 4 at North Florida Regional Hospital in Gainesville, Fla.). A scheduling representative at a second hospital also generates a similar list of work shifts that need to be filled for the upcoming time period at the second hospital.

The respective lists of upcoming work shifts for the first and second hospitals are then transmitted to the central scheduling server via the Internet (e.g., using a suitable API). The central scheduling server may also receive, from each of the first and second hospitals, an indication of how each of the first and second hospitals wishes to staff the work shifts, how each hospital is willing to compensate available independent health care workers for each work shift and/or type of work shift, etc.

For example, the first hospital and second hospital may both specify that: (1) on-staff physicians, advanced practitioners, and nurses are to be allowed to sign up for available shifts beginning on a first date (e.g., Jun. 1, 2017); (2) highly-rated independent-contractor physicians, advanced practitioners, and nurses (e.g., physicians, advanced practitioners, and nurses that have been ranked, within the system, with a rating above a particular level (e.g., four out of five stars)) are to be allowed to sign up for available shifts beginning on a second date (e.g., Jun. 6, 2017); and (3) all remaining candidate physicians, advanced practitioners, and nurses are to be allowed to sign up for any remaining available shifts beginning on a third date (e.g., Jun. 12, 2017). The hospital may further specify that the signup window for the scheduling period will close on a specific date (e.g., Jun. 30, 2017).

The first hospital may specify a first standard compensation level for each shift, and also specify circumstances under which healthcare workers may be compensated more or less than the standard compensation level for a particular shift. For example, the first hospital may specify that independent contractor healthcare workers who work overnight shifts will be paid 30% more than the standard compensation level for that type of shift. Similarly, the first hospital may specify that independent-contractor healthcare workers who currently have performance ratings above a particular rating level will be paid 15% more than other, similarly qualified independent-contractor heath care workers working the same shift. The second hospital may provide a similar compensation schedule.

Referring to FIG. 5A, assuming the first hospital account with the system 100 is set so that shifts are automatically scheduled based on one or more of the criteria discussed above (e.g., compensation, rating, etc.) the system 100 populates a calendar 502 of the shifts that have been scheduled and the shifts that are open 504. Open shifts 504 may be shown in a different color to distinguish them from assigned shifts. Referring to FIG. 5B, the system selects user availability that has been transmitted from users and looks for user availability that matches the open shifts 504 on the calendar 502. Referring to FIG. 5C, once the system 100 assigns an open shift 504 to the user, the system 100 send a notification 506 of the assignment. In various embodiments, a separate notification may be sent for each assigned work shift. In other embodiments, a single notification may be sent for all assigned work shifts to the user. Referring to FIG. 5D, once the open work shift has been assigned, the system shows the previously open work assignment as an assigned work assignment and indicates the name of the user assigned that work shift.

Referring to FIG. 6A, if the first business has their account set to manual assignment, then a scheduling assistant may manually assign open work shifts to users. For example, similar to FIG. 5A, a shift calendar 602 is created by the system 100. When the system receives a notification from a user of a request for open shifts, the notification is indicated by the system at 604. When the scheduling assistant selects the notification icon 604, a notification window 606 opens that displays the user's scheduling request. The scheduling assistant can select a “synch matched shifts” menu item 608 that places an availability overlay 610 for the user on the calendar display 602, as shown in FIG. 6B. Referring to FIG. 6B, the user availability in the availability overlay 610 is aligned with the shift calendar 602 to clearly display the user's availability with open shifts 612 shown on the calendar. Referring to FIG. 6C, the scheduling assistant can filter the shift calendar 602 so that it only shows the open shifts instead of both open shifts and assigned shifts. In the filtered view, the scheduling assistant may hover a pointer 614 over the assignment request date that aligns with an open work assignment 612 and drag the selected work box 616 into the open work shift, as shown in FIG. 6E. As a result, when the scheduling assistant switches from the filtered mode into the unfiltered mode shown in FIG. 6F, the calendar 602 shows all work shifts, including the newly assigned work shift 618.

As users sign up for various shifts, the central scheduling server may transmit updated respective scheduling information to each respective hospital scheduling server according to any appropriate schedule (e.g., in real time, nightly, weekly, etc.). For example, the central scheduling server may transmit updated scheduling information for the first hospital to the first hospital in real time so that the first hospital's scheduling server retains an up-to-date schedule of the work shifts. The central scheduling server may transmit updated scheduling information for the second hospital to the second hospital in real time so that the second hospital's scheduling server retains an up-to-date schedule of the work shifts.

At the closing date/time for signup for the particular scheduling time period, the system 100 may optionally generate and transmit a suitable electronic message to any participating users and/or hospitals indicating that the scheduling time period has closed. If any work shifts remain unfilled at that time: (1) the system may open an extended time period that may, for example, include additional incentives for signing up for the unfilled work shifts (e.g., a 10% compensation bonus for those work shifts); or (2) a suitable human representative may work to manually fill those positions (e.g., with staff healthcare workers).

Automated Swapping of Work Shifts Between Users

In various business settings, the swapping of work shifts between employees and contractors is a difficult task and may require the intervention of a third party to facilitate a work shift change such as human resources, a manager, a scheduler or another person. Thus, swapping work shifts becomes an arduous task that cannot be accomplished efficiently and on short notice. The scheduling system 100 described herein provides a quick and efficient method of automating the swapping of work shifts between users without the help of a third party.

Referring to FIG. 7A-7J, the system 100 allows a user to easily swap a work assignment with another user. Referring to FIG. 7A, the system presents a first user's work schedule via a user interface 702A. The schedule may be in a calendar form 704 where scheduled work days 706 are shown in gray, open days 708 are shown in white and requested work days 710 are shown in green. Referring to FIG. 7B, if the first user needs to swap a work shift with another user the first user may select the work shift 712, which becomes highlighted. Referring to FIG. 7C, after the work shift is selected, a user interface 702C opens that displays the details of the work shift and allows the first user to select an option 714 to request to swap the work shift. Referring to FIG. 7D, when the first user selects the option 714, a menu item 716 activates that allows the first user to select another provider (e.g., a second user) to which a work shift swap request will be sent. The system 100 may be configured to only list providers that are available to swap with the first user. Said another way, the system 100 may be configured to only list providers that do not have a work shift conflict with the work shift that the first user is trying to swap out. Referring to FIG. 7E, once the first user selects the second user and selects enter, an electronic notification 718 is sent to the selected second user. The electronic notification may be a text message with a link that opens a program, it may be a notification for the program, or it may be any other electronic notification.

Referring to FIG. 7F, when the second user selects the electronic notification 718, the second user is directed to a user interface 702F that provides an indication 720 to the second user that the first user is requesting a work shift swap. The user interface 702F also provides a selection menu item 722 that allows the second user to confirm whether they are willing to swap shifts. Referring to FIG. 7G, when the second user selects the menu item 722, a dropdown box 724 opens and allows the user to select either a “yes, I can swap a date” 726 or a “Sorry, I′m not available” 728. Referring to FIG. 7H, if the second user selects the “yes, I can swap a date” 726 from the drop down menu 722, the user interface 702F presents the second user with a “select a day from my calendar” menu items 728 that allows the second user to select a work shift to swap with the first user. Referring to FIG. 7I, when the user clicks on the “select a day from my calendar” menu items 728 (FIG. 7H), a calendar user interface 7021 opens to allow the user to select a work shift 730 from the calendar user interface 702I. Referring to FIG. 7J, once the second user selects the work shift 730 that they are willing to swap for the work shift that was requested by the first user, the second user can select a “send confirmation” menu item 732 on user interface 702 to send a swap confirmation back to the first user.

At this point, the system 100 updates the schedule to include that the second user is now working the shift on Jan. 20, 2017 and the first user is now scheduled to work the shift on Jan. 31, 2017. The system 100 may also be configured to transmit the shift changes back to the first hospital scheduling server so that the data at the first hospital is up to date. In various embodiments, the system 100 may be configured to allow the first user to put a time limit on the swap request that allows the request to be active for a certain period of time. In this way, if the second user does not respond to the swap request in a timely manner, the first user can send a new swap request to a different provider (e.g., a third user) all without having to manually track down the second user.

Manual Scheduling by Selected Providers

In yet another embodiment, the system 100 may be configured to allow a first business (e.g., a first hospital) to create a shift schedule and select one or more providers to schedule themselves in open shifts. For example, a hospital may fill open shifts by selecting preferred providers and allowing them to assign themselves to open shifts. The system may further be configured to allow the selected providers to select shifts all at the same time, provide certain providers first dibs on selecting shifts before the other providers (e.g., senior providers get first option before more junior providers, higher rated providers select before lower rated providers, etc.).

Referring to FIG. 8A, the scheduler for the first hospital has created a schedule 800 with an open shift 802. Referring to FIG. 8B, when the scheduler selects the open shift 802 on the schedule, an invite box 804 opens that allows the scheduler to select one or more users to fill the open shift in the schedule. The invite box 804 allows the schedule to select one or more users in a “select provider” drop down menu 806. Referring to FIG. 8C, the scheduler may pick one or more users by name or they may pick users based on groupings such as “all tiers”, “tier 1”, “tier 2”, “tier 3”, etc. Each tier can be based on one or more of a user's seniority level, a user's rating level, a compensation amount that the user has indicated that they want for working a shift, or any other suitable criteria that can be used to group providers. Additionally and referring to FIG. 8D, the invite box 804 includes a notes field 808 where the scheduler may include the compensation amount available to a user that works the open shift. The notes field may also contain any other information that is associated with the open shift. Once the scheduler is finished entering information in the invite box 804, the schedule can select an “send invite” menu item 810 to send the invite to the one or more selected providers.

Continuing with the above example and referring to FIG. 9A, once the scheduler sends the invite, one of the selected providers will receive an electronic notification 900 on their computing device (e.g., a smart phone, a tablet, a laptop, etc.). When the user selects the notification 900, it brings the user to a user interface 902B for the system 100 where the user can view the invite information. The invite may indicate the contact information 904 for the hospital administrator (e.g., scheduler, department manager, etc.), an offer time limit 906 (e.g., when the offer to work the open shift expires, and shift information 908. The user interface 902 may also provide a “sync this shift” menu item 910 that allows the system 100 to show the open shift in a calendar view 912 (FIG. 9C).

Referring to FIG. 9D, the user may filter the view in the schedule by selecting a “filter” menu item 914 (FIG. 9C) that opens a filter user interface 916. The user can then select a “open shifts” menu item 918 that overlays the user's open day 920 adjacent to and aligned with the calendar view 912. Thus, the user can easily compare their open days with the unassigned shifts available to them. Referring to FIG. 9F, the user can also select an “open shifts” menu item 922 using the filter 914 thereby simplifying the calendar view that is provided to the user, as shown in FIG. 9G. Thus, in the view shown in FIG. 9G, only the open shifts for the hospital are shown in aligned with the open dates for the user. In this example, the user may select open date 924 in FIG. 9G and drag it onto the unassigned shift (e.g., open shift) 926 for the hospital as shown in FIG. 9H. The system 100 may automatically update the schedule and present the updated calendar schedule view 912, as shown in FIG. 9I, with the user now assigned to the previously open shift 926 (FIG. 9G). In various embodiments, once a user assigns themselves to an open shift, the system updates the schedule and provides notice to other providers that may have been notified of the open shift. In this way, only one user can schedule themselves to the open shift.

Resource Marketplace

From the above description of the system and the use cases discussed herein, one of skill in the art should understand that the system 100 may be configured to create a marketplace for a geographic area (e.g., a city, a state, a region or the united states) for assigning resources (e.g., providers of services, people, equipment, etc.) to requesting parties. For example, in the case of health care workers, the system may allow health care providers from around the country to request work shifts from any organization that is seeking to fill unassigned work shifts. In addition, the system may also be configured to allow a user to request electronic credentialing at a particular facility (e.g., hospital, hospital group, facility, etc.) that is requesting a shift to be filled by providing information via the system 100 that is electronically shared with the facility. In various embodiments, the system 100 may automatically verify a health care providers license information, education, etc. through electronic state records and carry out the credentialing process on behalf of the facility. In this way, credentialing of health care providers is automated and streamlined eliminating the need to send papers through the mail and expediting the process so that healthcare providers can fill shifts on short notice. In other embodiments, the system may collect the necessary information from the healthcare provider, including uploaded support documentation, and allow the facility to verify the information. Once the facility credentials the healthcare provider, the system may update their electronic records and assign work shifts to the credentialed healthcare provider.

In other embodiments, the system may also be used as a resource marketplace for equipment. For example, business that is in need of specialized equipment may register with the system and upload request for equipment to the system 100. For example, if a business is in need of a specialized crane for a particular number of days at a particular location, the information can be loaded to the system and notification may be sent to equipment owners. The equipment owners may then evaluate the request, submit a bid including requested compensation for use of the equipment and the system can either automatically assign the owners bid to the request or allow the requester to manually assign the bid to the request. Once a bid is assigned to a request, the system would notify the bidder that the requester has accepted the bid.

### Conclusion

Many modifications and other embodiments of the invention will come to mind to one skilled in the art to which this invention pertains having the benefit of the teachings presented in the foregoing descriptions and the associated drawings. While examples discussed above cover the use of the invention in the context of scheduling work shifts in healthcare facilities, various embodiments may be used in any other suitable context (e.g., within the context of scheduling other types of work shifts, or within the context of scheduling other types of activities such as volunteers at a shelter, parents assigned tasks related to kids athletic programs, resources assigned in a school setting, etc.). Also, it should be understood that the system may be adapted to allow staff personnel (e.g., staff physicians, advanced practitioners, and/or nurses to sign up to work extra shifts for additional compensation beyond their salary (e.g., overtime). For example, the system may allow staff personnel to select to work extra shifts (e.g., along with independent-contractor physicians, advanced practitioners, and nurses) after the system has scheduled all available staff personnel to work full schedules. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for the purposes of limitation.

1. A computer-implemented data processing method for electronically scheduling workers to unassigned shifts based on various criteria associated with the unassigned shifts, the method comprising:
a. electronically receiving, by one or more computer processors a first set of scheduling data associated with a first hospital comprising unassigned work shifts that are available during a scheduling period;
b. identifying, by one or more computer processors, one or more users that meet particular criteria associated with each one of the unassigned work shifts in the first set of scheduling data;
c. providing notification to a first user of the one or more users, by one or more computer processors, that a first plurality of the unassigned work shifts are available for assignment;
d. at least partially in response to receiving a request from the first user to view the first plurality of unassigned work shifts, displaying, by one or more computer processors, the first plurality of unassigned work shifts to the first user;
e. receiving from the first user, by one or more computer processors, a first request to have one or more of the first plurality of unassigned work shifts assigned to the first user; and
f. in response to receiving the request, facilitating assignment of at least one of the one or more of the first plurality of unassigned work shifts to the first user.

2. The computer-implemented data processing method of claim 1, the method further comprising displaying to the first user, by one or more computer processors, an indication of compensation that the first user will receive if the first user completes one or more of the first plurality of unassigned work shifts.

3. The computer-implemented data processing method of claim 1, wherein the step of identifying one or more users that that meet particular criteria associated with each one of the unassigned work shifts in the first set of scheduling data further comprises:
a. determining, by one or more computer processors, the qualifications of each user;
b. comparing, by one or more computer processors, the qualifications of each user to the minimum qualifications required for each one of the unassigned work shifts in the first set of scheduling data; and
c. selecting, by one or more computer processors, one or more users to notify about the first plurality of the unassigned work shifts.

4. The computer-implemented data processing method of claim 1, wherein the step of providing notification to a first user that a first plurality of the unassigned work shifts are available for assignment further comprises determining, by one or more computer processors, whether to provide notification to the first user based, at least in part, on one or more work performance ratings for the first user.

5. The computer-implemented data processing method of claim 1, wherein the step of providing notification to a first user that a first plurality of the unassigned work shifts are available for assignment is based on one or more characteristics associated with the first user selected from a group consisting of:
a. the first user's work experience;
b. the first user's licensure;
c. ratings of the first user by their peers;
d. ratings of the first user by prior employers;
e. ratings of the first user by patients of the first user;
f. the first user's employment status; and
g. the first user's expected compensation.

6. The computer-implemented data processing method of claim 1, the method further comprising the steps of:
a. electronically receiving, by one or more computer processors, a second set of scheduling data associated with a second hospital comprising unassigned work shifts;
b. identifying, by one or more computer processors, one or more users that meet particular criteria associated with each one of the unassigned work shifts in the second set of scheduling data;
c. providing notification to the first user, by one or more computer processors, that a second plurality of the unassigned work shifts from the second set of scheduling data are available for assignment;
d. at least partially in response to receiving a request from the first user to view the second plurality of unassigned work shifts, displaying, by one or more computer processors, the second plurality of unassigned work shifts to the first user;
e. receiving from the first user, by one or more computer processors, a request to have one or more of the second plurality of unassigned work shifts assigned to the first user;
f. in response to receiving the second request, facilitating assignment of at least one or the one or more of the second plurality of unassigned work shifts to the first user.

7. The computer-implemented data processing method of claim 6, wherein displaying the first and second plurality of unassigned work shifts to the first user occurs substantially simultaneously.

8. The computer-implemented data processing method of claim 6, wherein the first request and the second request are the same request.

9. A computer-implemented data processing method for scheduling workers to unassigned work shifts, the method comprising:
a. electronically receiving, by one or more computer processors, a first set of scheduling data associated with a first business indicating a first plurality of unassigned work shifts and at least one criterion for each unassigned work shift;
b. displaying to a first user, by one or more computer processors, a first plurality of unassigned work shifts that are available to the first user;
c. receiving from the first user, by one or more computer processors, a first request to have one or more of the first plurality of unassigned work shifts assigned to the first user; and
d. in response to receiving the first request, facilitating the assignment of the one or more of the first plurality of unassigned work shifts to the user.

10. The computer-implemented data processing method of claim 9, the method further comprising:
a. receiving, by one or more processors, from a second business computer, a second set of scheduling data indicating a second plurality of unassigned work shifts;
b. displaying to the first user, by one or more processors, a second indication that the second plurality of unassigned work shifts are available for assignment;
c. receiving from the first user, by one or more processors, a second request to have one or more of the second plurality of unassigned work shifts assigned to the first user; and
d. in response to receiving the second request, facilitating the assignment of the one or more of the second plurality of unassigned work shifts to the first user.

11. The computer-implemented data processing method of claim 10, wherein
a. the method further comprises executing the step of displaying the first indication and the step of displaying the second indication simultaneously; and
b. the first request and the second request are the same request.

12. The computer-implemented data processing method of claim 10, the method further comprising determining whether to display the first set of scheduling data to the first user based, at least in part, on one or more work performance ratings for the first user.

13. The computer-implemented data processing method of claim 12, the method further comprising determining whether to display the second set of scheduling data to the user based, at least in part, on one or more work performance ratings for the first user.

14. The computer-implemented data processing method of claim 9, the method further comprising displaying to the first user, by one or more processors, an indication of specific compensation that the first user will receive if the user completes one or more of the unassigned work shifts.

15. The computer-implemented data processing method of claim 14, the method further comprising determining, by one or more processors, the specific compensation based, at least in part, on one or more work performance ratings for the first user.

16. The computer-implemented data processing method of claim 9, the method further comprising:
a. determining, by one or more processors, whether the first user is qualified to work the one or more work shifts; and
b. executing the displaying step at least partially in response to determining that the first user is qualified to work the one or more work shifts.

17. A computer-implemented data processing method for scheduling work shifts, the method comprising:
a. receiving, by one or more computer processors, from a first business computer, a first set of scheduling data comprising first unassigned work shifts for the first business;
b. providing notification to a first user, by one or more computer processors, that a first plurality of the first unassigned work shifts are available for assignment;
c. receiving from the first user, by one or more computer processors, a request to have one or more of the first plurality of first unassigned work shifts assigned to the first user, wherein the request comprises a compensation amount that the first user is willing to accept for each of the one or more of the first plurality of first unassigned work shifts; and
d. in response to receiving the request, facilitating assignment of the one or more of the first plurality of first unassigned work shifts at least partially based on the requested compensation amount.

18. The computer-implemented data processing method of claim 17, wherein the step of facilitating assignment of the one or more of the first plurality of first unassigned work shifts is further based on a rating associated with the first user.

19. The computer-implemented data processing method of claim 17, wherein the step of providing notification to a first user that a first plurality of the unassigned work shifts are available for assignment further comprises displaying, by one or more computer processors, the first plurality of first unassigned work shifts in a calendar display.

20. The computer-implemented data processing method of claim 19, wherein the calendar display is integrated with the first user's electronic calendar.

21. The computer-implemented data processing method of claim 17, the method further comprising:
a. receiving, by one or more computer processors, from a second business computer, a second set of scheduling data comprising second unassigned work shifts for the second business;
b. providing notification to the first user, by one or more computer processors, that a second plurality of the second unassigned work shifts are available for assignment;
c. receiving from the first user, by one or more computer processors, a second request to have one or more of the second plurality of second unassigned work shifts assigned to the first user, wherein the request comprises a compensation amount for each of the one or more of the second plurality of second unassigned work shifts; and
d. in response to receiving the second request, facilitating assignment of the one or more of the second plurality of second unassigned work shifts at least partially based on the requested compensation amount.
